(19) United States
(12) Patent Application Publication
(10) Pub. No.: US 2003/0069452 A1
(43) Pub. Date: Apr. 10, 2003

(54) METHOD AND APPARATUS FOR SYNTHESIZING FROM ALCOHOLS AND ETHERS FROM ALKANES, ALKENES, AND AROMATICS

(76) Inventors: Jeffrey H. Sherman, Sebastian, FL (US); Eric McFarland, Santa Barbara, CA (US)

Correspondence Address: Michael A. O'Neil, Michael A. O'Neil, P.C., Suite 1030, 5949 Sherry Lane, Dallas, TX, 75225, US

(21) Appl. No.: 10/298,440

(22) Filed: Nov. 19, 2002

Related U.S. Application Data
(63) Continuation-in-part of application No. 10/208,068, filed on Jul. 29, 2002, now abandoned.
Application No. 10/208,068 is a continuation-in-part of application No. 10/054,004, filed on Jan. 24, 2002, now Pat. No. 6,486,368.
Application No. 10/054,004 is a continuation-in-part of application No. 09/951,739, filed on Sep. 11, 2001, now Pat. No. 6,465,696.
Application No. 09/951,739 is a continuation-in-part of application No. 09/886,078, filed on Jun. 20, 2001, now Pat. No. 6,472,572.

(51) Int. Cl. C07C 27/10; C07C 35/08

(52) U.S. Cl. 568/694; 568/800; 568/822; 568/910

(57) ABSTRACT

A reactant selected from the group consisting of alkanes, alkenes, and aromatics is reacted with a metal halide to form the halide of the reactant and reduced metal. The reduced metal is oxidized to form metal oxide. The metal oxide is reacted with the halide of the reactant to produce the alcohol and/or the ether corresponding to the reactant and the original metal halide which is recycled.

### CROSS-REFERENCE TO RELATED APPLICATIONS

This application is a continuation-in-part of prior application Ser. No. 10/208,068, filed Jul. 29, 2002, currently pending, which is a continuation-in-part of prior application Ser. No. 10/054,004 filed Jan. 24, 2002, currently pending which is a continuation-in-part of prior application Ser. No. 09/951,739, filed Sep. 11, 2001, now U.S. Pat. No. 6,465,696, which is a continuation-in-part of application Ser. No. 09/886,078 filed Jun. 20, 2001, currently pending.

### TECHNICAL FIELD

This invention relates generally to synthesizing alcohols and ethers from alkanes, alkenes, and aromatics, and more particularly to a process wherein a reactant is reacted with a metal halide to form the halide of the reactant and reduced metal, wherein the reduced metal is oxidized to form metal oxide, and wherein the resulting metal oxide is reacted with the halide of the reactant to form the corresponding alcohol and/or ether.

### BACKGROUND AND SUMMARY OF THE INVENTION

This application is a continuation-in-part of co-pending application Ser. No. 10/208,068 filed Jun. 29, 2002, the disclosure of which is incorporated herein by reference as if fully set forth herein. The co-pending application discloses a process for converting ethane to diethyl ether, ethanol and ethyl acetate wherein ethane reacts with a halogen selected from the group including chlorine, bromine, and iodine. For example, ethane is reacted with bromine to form bromoethane and HBr. The bromoethane then reacts with metal oxide to form diethyl ether, ethanol, ethyl acetate, and metal bromide. The metal bromide reacts with oxygen or air to regenerate the original metal oxide. In the process, bromine and metal oxide are recycled.

The present invention comprises the method and apparatus for converting alkanes, alkenes, and aromatics to alcohols and/or ethers which differs substantially from the above described process. In accordance with the broader aspects of the invention, a reactant comprising an alkane, an alkene, and/or an aromatic is reacted with a metal halide to produce the halide of the reactant and reduced metal. The reduced metal is oxidized with air or oxygen to form the corresponding metal oxide. The metal oxide is reacted with the halide of the reactant to form the alcohol and/or the ether corresponding to the original alkane, alkene, or aromatic and the original metal halide which is recycled.

In accordance with a specific embodiment of the invention, ethane is reacted with metal bromide to form ethyl bromide and reduced metal. The reduced metal is reacted with oxygen and/or air to form the corresponding metal oxide. The metal oxide is reacted with the ethyl bromide to form ethanol and/or diethyl ether and metal bromide which is recycled.

In accordance with a particular aspect of the invention, steam is introduced into the ethyl bromide/metal oxide reaction to drive the reaction toward the production of ethanol. Alternatively, water can be removed from ethyl bromide/metal oxide reaction to drive the reaction toward the production of diethyl ether.

### BRIEF DESCRIPTION OF THE DRAWINGS

A more complete understanding of the invention may be had by reference to the following Detailed Description when taken in conjunction with the accompanying Drawings wherein:

FIG. 1 is a diagrammatic illustration of a first embodiment of the invention;

FIG. 2 is a diagrammatic illustration of a second embodiment of the invention;

FIG. 3 is a diagrammatic illustration of a third embodiment of the invention; and

FIG. 4 is a diagrammatic illustration of a fourth embodiment of the invention.

### DETAILED DESCRIPTION

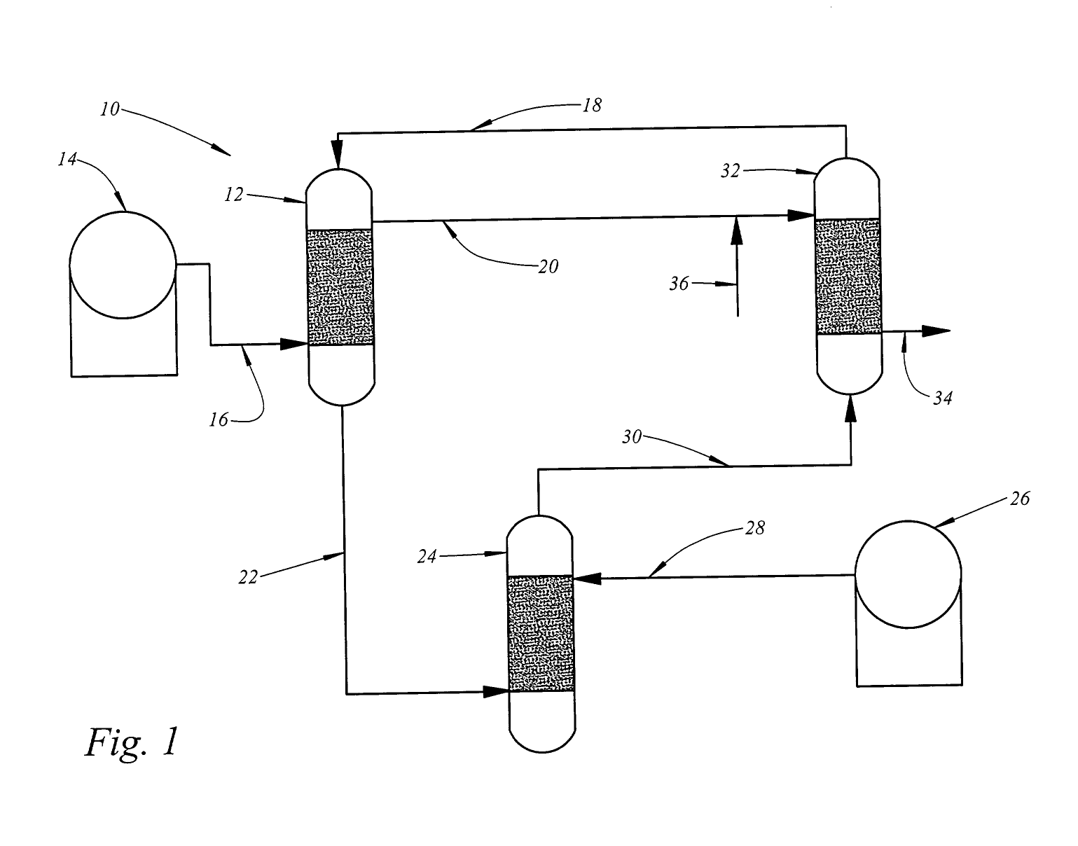

Referring now to the Drawings, and particularly to FIG. 1 thereof, there is shown a method and apparatus 10 for synthesizing alcohols and/or ethers comprising a first embodiment of the invention. The method and apparatus 10, as well as the other embodiments of the invention hereinafter described, can be used to synthesize alcohols and ethers from alkanes, alkenes, and/or aromatics. The following description wherein alcohols and/or ethers are synthesized from alkenes is representative.

A selected alkane, which may comprise methane, ethane, propane, butane, isobutane, pentane, hexane, cyclohexane, etc., is received in a first reactor 12 from a suitable source 14 through a line 16. The reactor 12 also receives a metal halide through a line 18. The halide comprising the metal halide that is received in the reactor 12 is selected from the group including chlorine, bromine, and iodine.

The reaction of the alkane with the metal halide produces the corresponding alkyl halide which is recovered through a line 20. The reaction also produces metal in reduced form, such as a metal hydride, which is recovered through a line 22 and directed to a second reactor 24. The second reactor 24 also receives oxygen and/or air from a source 26 through a line 28.

The second reactor 24 functions to convert the reduced metal received through the line 22 to metal oxide which is recovered through a line 30 and directed to a third reactor 32. The temperature of the reaction within the second reactor 24 is low enough that any bromine remaining on the metal following the reaction in the first reactor 12 remains on the metal, and only the hydrogen on the metal is replaced with oxygen. The hydrogen liberated from the metal is converted to water.

Within the third reactor 32 the alkyl halide formed in the first reactor 12 reacts with the metal oxide formed in the second reactor 24 to form the corresponding alcohol and/or ether which is recovered through an outlet 34. The reaction in the third reactor 32 also produces metal halide which is recycled to the first reactor 12 through the line 18.

In accordance with a specific application of the invention, the first reactor 12 receives ethane from the source 14 through the line 16, and the metal halide received in the reactor 12 through the line 18 comprises metal bromide. The reaction within the reactor 12 produces ethyl bromide which is recovered through the line 20 and directed to the third reactor 32. The reaction within the third reactor 32 may be characterized as follows:

4CH3CH2Br+Metal Oxide+XH2O→2CH3CH2OH+CH3CH2OCH2CH3+Metal Bromide2

As will therefore be apparent, the reaction within the reactor 32 can be biased toward the production of alcohol or toward the production of ether depending upon the amount of water present within the reactor. FIG. 1 further illustrates an operational mode of the first embodiment of the invention whereby steam is directed into the line 20 through a line 36 and enters the reactor 32 with the ethyl bromide and the HBr that were produced in the reactor 12. As will be apparent from the formula set forth above, the introduction of steam into the reactor 32 causes the reaction therein to be selective toward the production of ethanol in the reactor 32 as opposed to the production of diethyl ether.

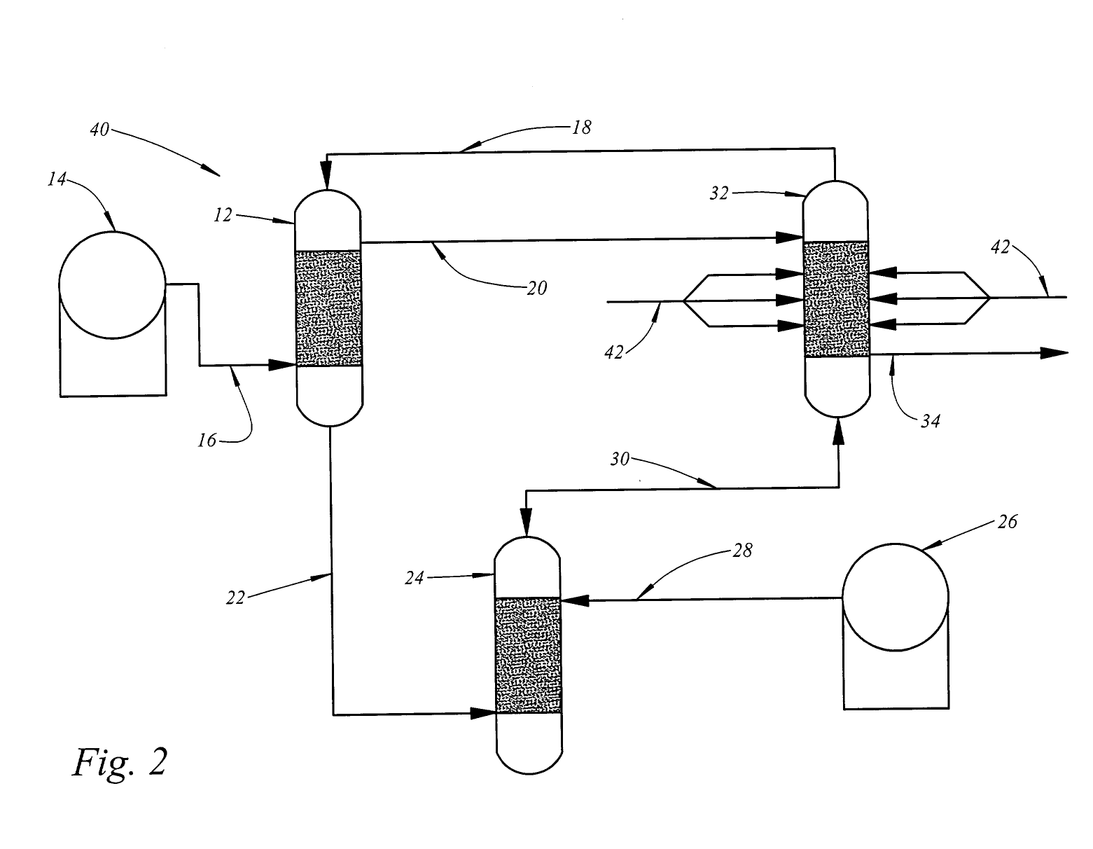

Referring now to FIG. 2, there is shown a method of and apparatus for producing alcohols and/or ethers from alkanes, alkenes, or aromatics comprising a second embodiment of the invention. Many of the components parts of the second embodiments of the invention are identical in construction and function to component parts of the first embodiment of the invention as illustrated in FIG. 1 and described hereinabove in conjunction therewith. Such identical component parts are designated in FIG. 2 with the same reference numerals utilized in the description of the first embodiment of the invention.

The second embodiment of the invention differs from the first embodiment of the invention in that rather than being directed into the reactor 32 through the line 20, steam is injected directly into the reactor 32 through lines 42. This allows the steam to be added to the reactor 32 at specific points in the reactor thereby increasing the selectivity of the reaction that takes place therein towards the production of alcohol and away from the production of ether.

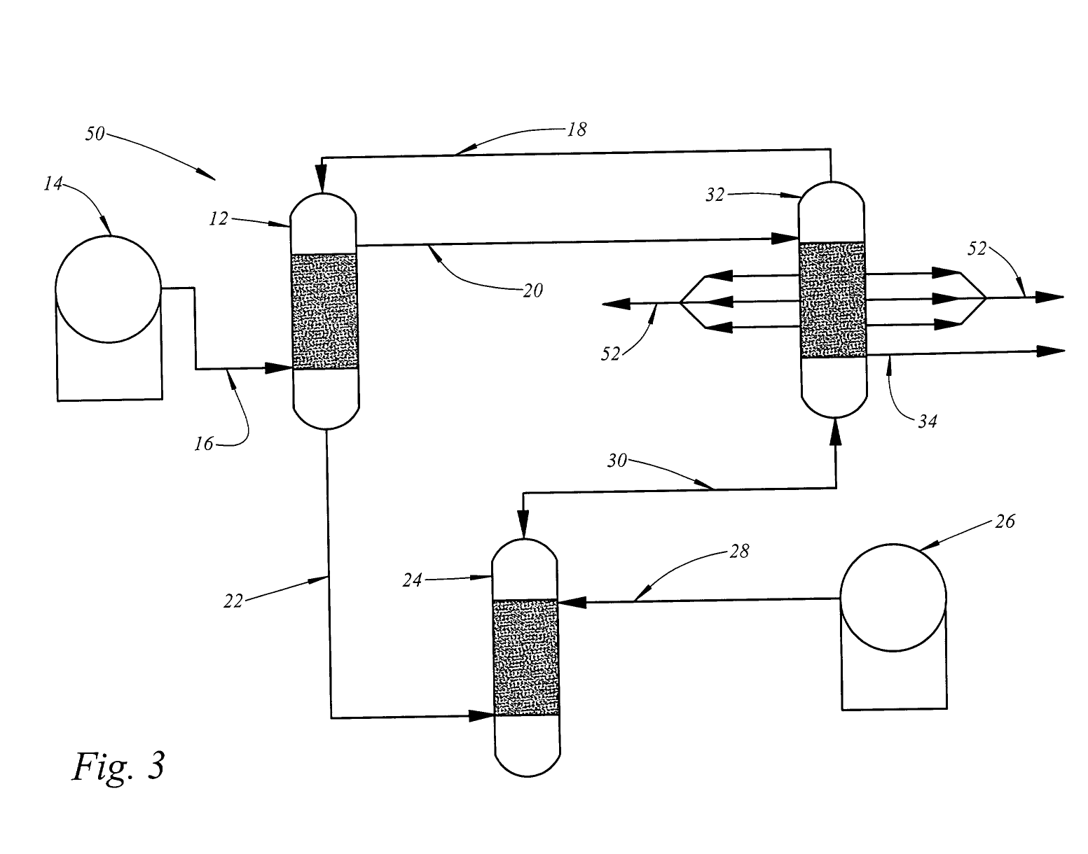

A method and apparatus 50 for producing alcohols and/or ethers from alkanes, alkenes, and aromatics comprising a third embodiment of the invention is illustrated in FIG. 3. Many of the component parts of the third embodiment of the invention are identical in construction and function to component parts of the first embodiment of the invention which are illustrated in FIG. 1 and described hereinabove in conjunction therewith. Such identical component parts are designed in FIG. 3 with the same reference numerals utilized in the description of the first embodiment of the invention.

The third embodiment of the invention differs from the first and second embodiments in that during the operation thereof water is removed from the reactor 32 through lines 52. The removal of water from the reactor 32 is accomplished by either distillation or by osmosis or by both. As will be apparent from the reaction set forth above, the removal of water from the reaction 32 causes the reaction that takes place therein to be selective toward the production of ether as opposed to the production of alcohol.

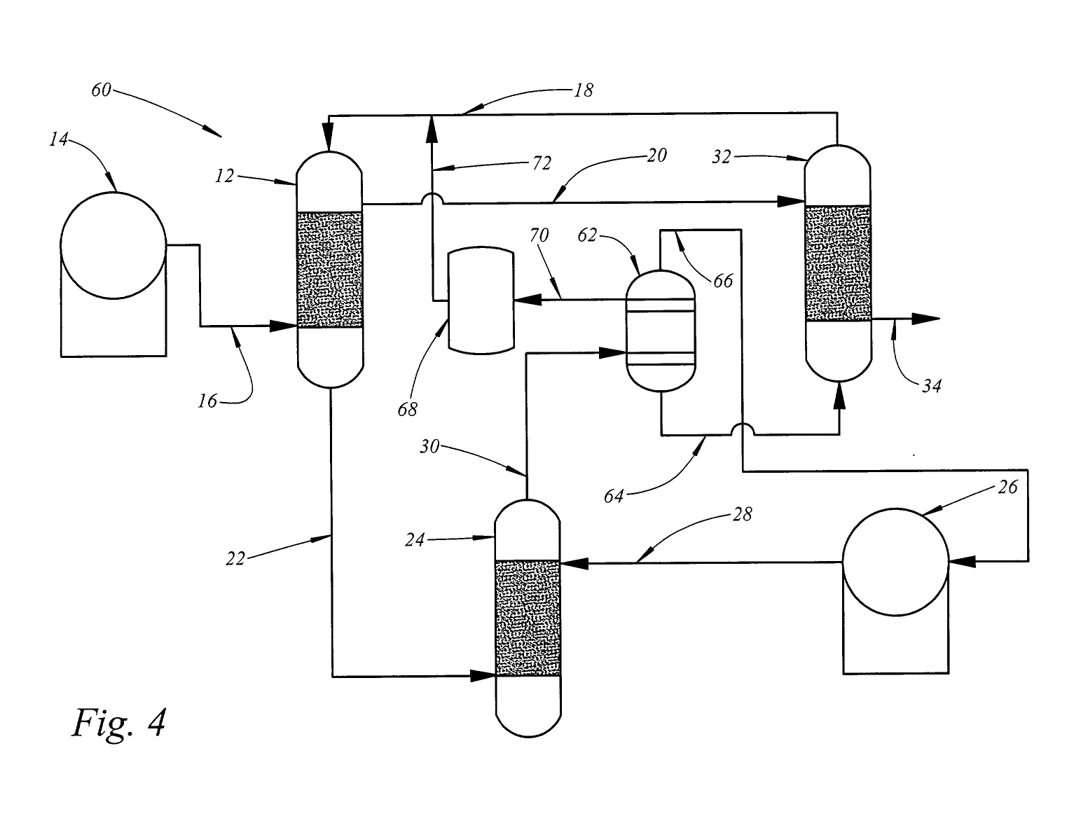

Referring now to FIG. 4,there is shown a method and apparatus 60 for producing alcohols and/or ethers from alkanes, alkenes, and aromatics comprising a fourth embodiment of the invention. Many of the component parts of the fourth embodiment of the invention are identical in construction and function to components of the first embodiment of the invention as illustrated in FIG. 1 and described hereinabove in conjunction therewith. Such identical components are designated in FIG. 4 with the same reference numerals utilized in the description of the first embodiment of the invention.

In accordance with the fourth embodiment of the invention, oxygen and/or air is directed into the second reactor 24 at a rate such that in addition to oxidizing the reduced metal back to metal oxide, additional oxygen is added to the metal oxide and molecular halide is liberated. The reaction products from the second reactor 34 are directed to a separator 62. The separator 62 directs metal oxide to the reactor 32 through a line 64, returns oxygen to the source 26 through a line 66 and directs halide to a halide storage tank 68 through a line 70. From the storage tank 68, the halide is directed to the reactor 12 through a line 72 and the line 18 thereby assuring an optimum level of halide within the reactor 12 at all times.

Although preferred embodiments of the invention have been illustrated in the accompanying Drawings and described in the foregoing Detailed Description, it will be understood that the invention is not limited to the embodiments disclosed but is capable of numerous rearrangements, modifications, and substitutions of parts and elements without departing from the spirit of the invention.

1. The method of synthesizing alcohols and/or ethers comprising the steps of:
a. providing a reactant selected from the group comprising alkanes, alkenes, and aromatics;
b. providing a metal halide;
c. reacting the metal halide with the reactant to produce the halide of the reactant and reduced metal;
d. reacting the reduced metal with an oxidizer to produce metal oxide;
e. reacting the metal oxide produced in step d. with the halide of the reactant produced in step c. to produce the alcohol and/or the ether corresponding to the reactant and the original metal halide; and
f. recycling the metal halide.

2. The method according to claim 1 wherein the reactant comprises an alkane selected from the group comprising methane, ethane, propane, butane, isobutane, pentane, hexane, and cyclohexane.

3. The method according to claim 1 wherein the metal halide of step b. comprises a halide selected from the group including chlorine, bromine, and iodine.

4. The method according to claim 1 wherein the oxidizer of step d. is selected from the group including air and oxygen.

5. The method according to claim 1 including additional step of adding steam to the reaction of step e. and thereby increasing the selectivity of the reaction to the synthesis of alcohol.

6. The method according to claim 1 including the additional step of removing water from the reaction of step e. and thereby increasing the selectivity of the reaction to the production of ether.

7. The method of claim 1 wherein the reactant of step a. comprises an alkane and wherein the halide comprising the metal halide of step b. is selected from the group consisting of chlorine, bromine, and iodine.

8. The method of claim 1 wherein the reactant of step a. comprises ethane, wherein the metal halide of step b. comprises metal bromide, and wherein the halide of the reactant of step c. comprises bromoethane.

9. A method of synthesizing alcohols and ethers comprising the steps of:
a. providing a first reactor;
b. providing a quantity of a reactant selected from the group consisting alkanes, alkenes, and aromatics;
c. providing a metal halide;
d. directing the reactant into the first reactor;
e. simultaneously directing the metal halide into the first reactor for reaction with the reactant to produce the halide of the reactant and reduced metal;
f. providing the second reactor;
g. providing a quantity of an oxidizer;
h. directing the reduced metal produced in step e. into the second reactor;
i. simultaneously directing the oxidizer into the second reactor for reaction with the reduced metal to reduce metal oxide;
j. directing the metal oxide produced in step i. into the third reactor;
k. directing the halide of the reactant produced in step e. into the third reactor for reaction with the metal oxide to produce the alcohol and/or the ether corresponding to the reactant and the original metal halide;
l. recovering the alcohol and/or the ether from the third reactor;
m. directing the metal halide produced in step k. from the third reactor to the first reactor.

10. The method according to claim 9 wherein the reactant comprises an alkane selected from the group comprising methane, ethane, propane, butane, isobutane, pentane, hexane, and cyclohexane.

11. The method according to claim 9 wherein the metal halide comprises a halide selected from the group including chlorine, bromine, and iodine.

12. The method according to claim 9 wherein the oxidizer of step g. is selected from the group including air and oxygen.

13. The method according to claim 9 including additional step of adding steam to the reaction of step k. and thereby increasing the selectivity of the reaction to the synthesis of alcohol.

14. The method according to claim 9 including the additional step of removing water from the reaction of step k. and thereby increasing the selectivity of the reaction to the production of ether.

15. The method of claim 9 wherein the reactant of step b. comprises an alkane and wherein the halide comprising the metal halide of step c. is selected from the group consisting of chlorine, bromine, and iodine.

16. The method of claim 9 wherein the reactant of step b. comprises ethane, wherein the metal halide of step c. comprises metal bromide, and wherein the halide of the reactant of step e. comprises bromoethane.

17. The method of claim 9 wherein the reaction of step k. is carried out at a temperature low enough that any halide remaining on the metal following the reaction in the first reactor remains on the metal following the reaction in the second reactor.

18. The method of claim 9 wherein the oxidizer of step g. is directed into the second reactor of step f. at a rate sufficient to liberate any halide remaining on the metal following the reaction in the first reactor.

19. The method of claim 18 including the additional step of separating the halide from any residual oxidizer following the reaction in the second reactor.

20. The method of claim 19 including the additional step of directing the halide that was liberated in the reaction in the second reactor to the first reactor.
